(19) United States
(12) Patent Application Publication
(10) Pub. No.: US 2021/0330583 A1
(43) Pub. Date: Oct. 28, 2021

(54) Promoting and enabling for the growth of more hair volume with a lotion made from the radish, garlic, and vitamin E oil combined at room temperature.

(71) Applicant: Marilyn Hsumei Chen, Phoenix, AZ (US)

(72) Inventor: Marilyn Hsumei Chen, Phoenix, AZ (US)

(21) Appl. No.: 15/929,342

(22) Filed: Apr. 27, 2020

(51) Int. Cl. A61K 9/00; A61K 36/8962; A61K 36/31; A61K 31/355; A61K 9/06

(52) U.S. Cl. CPC A61K 9/0014; A61K 36/8962; A61K 9/06; A61K 31/355; A61K 36/31

(57) ABSTRACT

The task of initiating a 20 minutes cycle of skin temperature regulation through Gibs Free Energy and sets in the breaking down of the nutrients for skin absorption by taking advantage of assimilating the mechanism of sebaceous gland's function in relation to the growth and maintenance of hair with radish, garlic, and vitamin E oil.

### BACKGROUND OF THE INVENTION

The reference of facts and most of the information came from Wikipedia except facts concerning the object of this invention.

This product has a garlic odor that is fading and turning into a soybean like odor throughout the application and lasted 24 hours. Storage is at room temperature with a 44 F* 24 hours prior refrigeration.

The adverse effects and toxicity of garlic after it is steam heated for 20 minutes has significantly minimized.

List of Nutrients Benefits:

Thiamine is important in carbohydrate metabolism. Its phosphorites derivate TPP is a coenzyme in the metabolism.

Riboflavin is an antioxidant for healthy skin and aids in metabolizing carbohydrates. It is required by the body for cellular respiration.

Pantothenic is that help in release energy from the foods.

Vitamin B6 help form proteins, and help carbohydrates metabolized.

Folates simulate hormones. And, it is necessary for the production and maintenance of new cells.

Vitamin C synthesize and metabolize coenzyme A. It is an essential nutrient involved in the repair of tissue as an antioxidant.

Calcium is a stabilizer of proteins including enzymes in optimizing their activities. A slight increase in blood calcium concentration stimulate the production and secretion of the peptide hormone.

Vitamin E is an antioxidant protecting cell membranes from Reactive Oxygen Species, a natural byproduct of normal metabolism of oxygen.

Phosphorus is a component of DNA, ATP.

Zinc is and antioxidant protect cells from damage.

Lists of Nutrients Noted Side Effects:

Thiamine (B1) could cause itching of skin, swelling of eye lid, wheezing, and hive.

Riboflavin (B2) has no evidence for excessive intake toxicity.

Niacin (B3) at low doses of 50-500 mg causing flushing, headache, abdominal pain, diarrhea, dyspepsia, nausea, vomiting, rhinitis, pruritus, and rash.

Pantothenic acid (B3) have no reported side effects.

Vitamin B6's adverse effects have been documented from B6 supplements. But, never from food sources.

Folates have no known adverse reaction topically.

Vitamin C exhibits low acute toxicity. Symptoms reported for large doses include nausea, abdominal cramps, and diarrhea.

Vitamin E when ROS's level increased dramatically may result in significant damage to cell structure.

Calcium's long-term concerns about its' adverse side effects including calcification of arteries and kidney stores.

Iron could cause allergic reaction of rash itching, dizziness, trouble breathing.

Magnesium has no documentation.

Phosphorus has no documentation.

Sodium when contacted with moisture on skin eyes, or mucous membranes can cause server burns.

Zinc can cause lowered immune function reduce sense of taste, smell, toxicity.

Selenium has no documentation.

Nutritional value per 100 g (3.5 oz) 3 ug of Garlic:

| Category | Value |
|---|---|
| Energy | 623 kj |
| Carbohydrates | 33.06 g |
| Sugars | 1 g |
| Dietary fiber | 2.1 g |
| Fat | 0.5 g |
| Protein | 6.36 g |
| Thiamine (B3) | 0.2 mg, 17% DV |
| Riboflavin (B2) | 0.11 mg, 9% DV |
| Niacin (B3) | 0.7 mg, 5% DV |
| Pantothenic acid | 0.596 mg, 12% DV |
| Vitamin B6 | 1.235 mg, 5% DV |
| Folates | 3 ug, 1% DV |
| Vitamin C | 31.2 mg, 38% DV |
| Calcium | 181 mg, 18% DV |
| Iron | 1.7 mg, 13% DV |
| Magnesium | 25 mg, 7% DV |
| Phosphorus | 153 mg, 22% DV |
| Potassium | 401 mg, 9% DV |
| Sodium | 17 mg, 1%DV |
| Selenium | 14.2 ug |
| Zinc | 1.16 mg, 12% DV |
| Water | 59 g |

Nutritional values per 100 g (3.5 oz) 3 ug of radish:

| Category | Value |
|---|---|
| Energy | 66 Kj |
| Carbohydrates | 3.4 g |
| Sugar | 1.86 g |
| Dietary fiber | 1.6 g |
| Fat | 0.1 g |
| Protein | 0.68 g |
| Thiamine (B1) | 0.012 mg, 1% DV |
| Riboflavin (B2) | 0.039 mg, 3% DV |
| Niacin (B3) | 0.254 mg, 2% DV |
| Pantothenic acid | 0.165 mg, 3% DV |
| Vitamin B6 | 0.165 mg, 5% DV |
| Folates | 25 ug, 6% DV |
| Vitamin C | 14.8 mg, 18% DV |
| Calcium | 25 mg, 3% DV |
| Iron | 0.34 mg, 18% DV |
| Magnesium | 10 mg, 3% DV |
| Phosphorus | 20 mg, 3% DV |
| Potassium | 233 mg, 5% DV |
| Zinc | 0.28 mg, 3% DV |
| Water | 95.3 g |

### BRIEF SUMMARY OF INVENTION

Since sebaceous glands help maintaining the integrity of the skin barriers and supply lipid to the skin. This product also benefiting by using vitamin E oil to protect water loss of skin.

As the problem of not having hair to facilitate the temperature changes, the scalp couldn't have cushioned to the extreme of its environments.

This lotion with the application of radish, garlic, and vitamin E oil help the skin regulate itself the temperature by applying topically at water temperature of 44 F*˜55 F* of this lotion product. And the supplying of vitamin E oil to protect epidermis layer of the skin reducing transdermal water loss from the heat loss and strengthens the skin's barriers function with the purpose of an antioxidant forming Reactive Oxygen Species as from normal metabolism of oxygen a and affecting cell signaling and homeostasis.

### DETAILED DESCRIPTION OF THE INVENTION

Stress cause hair loss. Thermoregulatory role of the hair needs lubrication and water proofing of the skin.

Upon the application of radish, garlic, and vitamin E oil combination, the skin is cool down with a temperature of 44 F*˜55 F* of this lotion leaving it on the skin at room temperature for 20 minutes, it will form a transparent thin film. Applicable method is to take 0.07 g of the lotion and apply on the scalp where it needs more volume and regrowth. Rinse with lukewarm water afterwards.

The initiated thermoregulation is formed using the Gibs Free Energy extracted from this thematically closed system.

This lotion is made in unit of a 230 g of raw radish, a 14 g of heated garlic, and a 21 g of vitamin E oil. Available for use after a period of 24 hours of refrigeration at 44 F*.

The growth of vellus looked alike hair is at normal of 1 mm per first 28 days. And, vellus hair could last more than six months. The regeneration of hair and its natural color is achieved with the continuing application of this lotion.

This lotion is benefiting by keeping scalp properly supplied with nutrients. As garlic yields enzymes which increase the reaction rate, and with that said, would bring vitamin E oil to a more soluble state. Vitamin E is a fa-soluble antioxidant protecting cell membranes from Reactive Oxygen Species.

The benefit of radish, an Amino Acid, is the release of a water molecule during the formation of each Amino Bond presented by Organic Addition Reaction.

1. The claimed invention is when one of the functions of hair is to keep cool from heat and wade cold for warmth, hair's growth and maintenance could be supported by assimilation of the existence of hair in the case of that it was lost on the scalp. So, the application is to support nutrients from garlic, radish, and vitamin E oil for absorbing through the method of Gibs Free Energy.
The mechanism is of that hair follicle regulates hair growth via interaction between hormones, neuropeptides and immune cells. Evidence suggests that nervous and immune systems have regulatory interaction with the skin.
This product's one of the three ingredients vitamin E is a group of eight fat soluble compounds disperse and store in fat for maintenance and growth.
Sebum is made of triglycerides, wax esters, and squalene metabolites. As sebum waterproofs and lubricates the skin at skin PH of 4.5 PH˜6.2 PH while the PH of garlic is 5.8 PH and radish at 5.8 PH and radish at 5.8 PH˜6.05 PH, sebaceous glands secreted acids and supplied sebum to the skin.
